(19) United States
(12) Patent Application Publication
(10) Pub. No.: US 2008/0027211 A1
(43) Pub. Date: Jan. 31, 2008

(54) Enantiopure Heterocyclic Compound Useful for the Preparation of Peptides Which Can Be Potentially Used as Medicaments

(75) Inventors: Roland Callens, Grimbergen (BE); Cyrille Pousset, Bruxelles (BE)

Correspondence Address: CONNOLLY BOVE LODGE & HUTZ, LLP, P O BOX 2207, WILMINGTON, DE, 19899, US

(73) Assignee: SOLVAY S.A., Brussels (BE)

(21) Appl. No.: 11/664,571

(22) PCT Filed: Oct. 4, 2005
(86) PCT No.: PCT/EP05/54982

§ 371 Date: Apr. 3, 2007

(30) Foreign Application Priority Data
Oct. 4, 2004 (FR) 04.10474

(51) Int. Cl. C07K 2/00; C07D 207/26; C07D 263/06; C07D 265/06; C07D 267/06; C07K 5/078; C07D 267/10; C07D 279/12; C07D 403/06

(52) U.S. Cl. 530/300; 540/200; 540/531; 540/544; 544/105; 544/171; 544/384; 544/51; 544/54; 544/58.1; 544/88; 546/174; 546/243; 546/248; 546/341; 548/146; 548/215; 548/217; 548/319.5; 548/324.1; 548/452; 548/551; 548/565; 548/572

(57) ABSTRACT

Enantiopure heterocyclic compound Enantiopure heterocyclic compound of formula (I) in which J is chosen from C, N, O and S; Z is H or a group for protecting the amino functional group, R₃ denotes H or an organic residue, m is 0, 1 or 2 and n is 0, 1 or 2, and in which the heterocycle is preferably substituted with at least one substituent other than CH₂—COOR₃.

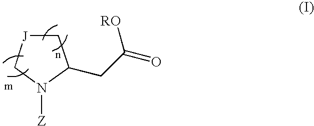

The present invention relates to enantiopure heterocyclic compounds.

Some β-amino acids and their derivatives are useful in the context of the manufacture of peptides which can be used as medicaments. Specific examples of such β-amino acids comprise at least one nitrogen-containing heterocycle.

In the search for active ingredients, it is desirable to have available amino acids which contribute to pharmacological activity, in particular peptides, peptide analogues or peptidomimetics, and which can be used in the process for manufacturing peptides or peptide analogues.

Patent U.S. Pat. No. 3,891,616 describes some biologically active peptides containing 2-pyrrolidineacetic acid. The N-Boc derivative of this acid is prepared by treating natural L-proline with diazomethane.

The invention aims to make available compounds which are useful in the context of the manufacture of peptides which can be potentially used as medicaments.

The invention therefore relates to an enantiopure heterocyclic compound of formula (I) 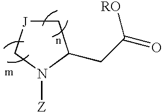
in which J is chosen from C, N, O and S; Z is H or a group for protecting the amino functional group, R denotes H or an organic residue, m is 0, 1 or 2 and n is 0, 1 or 2, and in which the heterocycle is preferably substituted with at least one substituent other than CH₂—COOR.

The expression enantiopure compound is understood to mean a chiral compound mainly consisting of one enantiomer. The enantiomeric excess (ee) is defined: ee (%)=100(x₁−x₂)/(x₁+x₂) with x₁>x₂; x₁ and x₂ represent the content of the enantiomer 1 or 2 respectively in the mixture.

The expression “organic residue” is understood to mean in particular linear or branched alkyl or alkylene groups which may contain heteroatoms such as in particular boron, silicon, nitrogen, oxygen and sulphur atoms, cycloalkyl groups, heterocycles and aromatic systems. The organic residue may contain double or triple bonds and functional groups.

The organic residue comprises at least I carbon atom. Often, it comprises at least 2 carbon atoms. Preferably, it comprises at least 3 carbon atoms. In a more particularly preferred manner, it comprises at least 5 carbon atoms.

The organic residue generally comprises at most 100 carbon atoms. Often, it comprises at most 50 carbon atoms. Preferably, it comprises at most 40 carbon atoms. In a more particularly preferred manner, it comprises at most 30 carbon atoms.

The expression “alkyl group” is understood to mean in particular a linear or branched alkyl substituent comprising from 1 to 20 carbon atoms, preferably 1, 2, 3, 4, 5, 6, 7, 8, 9 or 10 carbon atoms. Specific examples of such substituents are methyl, ethyl, n-propyl, isopropyl, n-butyl, iso-butyl, tert.-butyl, n-pentyl, isopentyl, n-hexyl, 2-hexyl, n-heptyl, n-octyl and benzyl.

The expression “cycloalkyl group” is understood to mean in particular a substituent comprising at least one saturated carbocycle of 3 to 10 carbon atoms, preferably 5, 6 or 7 carbon atoms. Specific examples of such substituents are cyclopropyl, cyclobutyl, cyclopentyl, cyclohexyl and cycloheptyl.

The expression “alkylene group” or “cycloalkylene group” is understood to mean in particular the bivalent radicals derived from alkyl or cycloalkyl groups as defined above.

When the organic residue contains one or optionally more double bonds, it is often chosen from an alkenyl or cycloalkenyl group comprising from 2 to 20 carbon atoms, preferably 2, 3, 4, 5, 6, 7, 8, 9 or 10 carbon atoms. Specific examples of such groups are vinyl, 1-allyl, 2-allyl, n-but-2-enyl, isobutenyl, 1,3-butadienyl, cyclopentenyl, cyclohexenyl and styryl.

When the organic residue contains one or optionally more triple bonds, it is often chosen from an alkinyl group comprising from 2 to 20 carbon atoms, preferably 2, 3, 4, 5, 6, 7, 8, 9 or 10 carbon atoms. Specific examples of such groups are ethinyl, 1-propinyl, 2- propinyl, n-but-2-inyl, and 2-phenylethinyl.

When the organic residue contains one or optionally more aromatic systems, it is often an aryl group comprising from 6 to 24 carbon atoms, preferably from 6 to 12 carbon atoms. Specific examples of such groups are phenyl, 1-tolyl, 2-tolyl, 3-tolyl, xylyl, 1-naphthyl and 2-naphthyl.

The expression “heterocycle” is understood to mean in particular a cyclic system comprising at least one saturated or unsaturated ring formed of 3, 4, 5, 6, 7 or 8 atoms of which at least one is a heteroatom. The heteroatom is often chosen from B, N, O, Si, P and S. More often, it is chosen from N, O and S.

Specific examples of such heterocycles are aziridine, azetidine, pyrrolidine, piperidine, morpholine, 1,2,3,4-tetrahydroquinoline, 1,2,3,4-tetrahydroisoquinoline, perhydroquinoline, perhydroisoquinoline, isoxazolidine, pyrazoline, imidazoline, thiazoline, tetrahydrofuran, tetrahydrothiophene, pyran, tetrahydropyran and dioxane.

The organic residues as defined above may be unsubstituted or substituted with functional groups. The expression functional group is understood to mean in particular a substituent comprising or consisting of one heteroatom. The heteroatom is often chosen from B, N, O, Al, Si, P, S, Sn, As and Se and the halogens. More often, it is chosen from N, O, S and P, in particular N, O and S.

The functional group generally comprises 1, 2, 3, 4, 5 or 6 atoms.

As functional groups, there may be mentioned for example halogens, a hydroxyl group, an alkoxy group, a mercapto group, an amino group, a nitro group, a carbonyl group, an acyl group, an optionally esterified carboxyl group, a carboxamide group, a urea group, a urethane group and the thiolated derivatives of the groups containing a carbonyl group which are mentioned above, phosphine, phosphonate and phosphate groups, a sulphoxide group, a sulphone group, a sulphonate group.

In the compounds according to the invention, the substituent Z in the compound of general formula (I) is often a group for protecting the amino functional group. These compounds may be used as they are as intermediate for peptide synthesis.

As nonlimiting examples of groups for protecting the aminofunctional group which may be represented by Z, there may be mentioned in particular groups of the alkyl or aralkyl type, which are substituted or unsubstituted, such as the benzyl, diphenylmethyl, di(methoxyphenyl)methyl or triphenylmethyl (trityl) group, groups of the acyl type, which are substituted or unsubstituted, such as the formyl, acetyl, trifluoroacetyl, benzoyl or phthaloyl group, groups of the aralkyloxycarbonyl type, which are substituted or unsubstituted, such as the benzyloxycarbonyl, p-chlorobenzyloxycarbonyl, p-bromobenzyloxycarbonyl, p-nitrobenzyloxycarbonyl, p-methoxybenzyloxycarbonyl, benzhydryloxycarbonyl, 2-(p-biphenyl)isopropyloxycarbonyl, 2-(3,5-dimethoxyphenyl)-isopropyloxycarbonyl, p-phenylazobenzyloxycarbonyl, triphenylphosphonoethyloxycarbonyl or 9-fluorenylmethyloxycarbonyl group, groups of the alkyloxycarbonyl type, which are substituted or unsubstituted, such as the tert-butyloxycarbonyl, tert-amyloxycarbonyl, diisopropylmethyloxycarbonyl, isopropyloxycarbonyl, ethyloxycarbonyl, allyloxycarbonyl, 2-methylsulphonylethyloxycarbonyl or 2,2,2-trichloroethyloxycarbonyl group, groups of the cycloalkyloxycarbonyl type, such as the cyclopentyloxycarbonyl, cyclohexyloxycarbonyl, adamantyloxycarbonyl or isobornyloxycarbonyl group, groups containing one heteroatom such as the benzenesulphonyl, p-toluenesulphonyl (tosyl), mesitylenesulphonyl, methoxytrimethylphenylsulphonyl, o-nitrophenylsulphenyl or trimethylsilane group.

Among these groups Z, those comprising a carbonyl group are preferred. The acyl, aralkyloxycarbonyl and alkyloxycarbonyl groups are more particularly preferred.

Preferably, the protecting group is sterically bulky. The expression “sterically bulky” is understood to mean in particular a substituent comprising at least 3 carbon atoms, in particular at least 4 carbon atoms of which at least one is a secondary, tertiary or quaternary carbon atom. Often, the sterically bulky group comprises at most 100, or even 50, carbon atoms. A protecting group chosen from alkoxycarbonyl, aryloxycarbonyl or aralkoxycarbonyl groups is preferred. A tert-butyloxycarbonyl (BOC) group is most particularly preferred.

In the compound according to the invention, J is advantageously chosen from N, O and S. Preferably, J is chosen from O and S.

In the compound according to the invention, the following combinations of n and m are possible: m=0 and n=0; m=1 and n=0; m=2 and n=0; m=l and n=1; m=1 and n=2; m=2 and n=1; m=2 and n=2. In a particular embodiment, m is 1 or 2 and n is 0 or 1.

In a first particular embodiment, the values of m and n correspond to any of the abovementioned combinations and J is N.

In a second particular embodiment, which is preferred, the values of m and n correspond to any of the abovementioned combinations and J is O.

In a third particular embodiment, which is preferred, the values of m and n correspond to any one of the abovementioned combinations and J is S.

The absolute configuration of the stereogenic centre which is necessarily present in the compound of formula (I) ((α- in relation to the nitrogen) is (R) or (S), each of the enantiomers being accessible and capable of being used as potentially biologically active ingredient or as intermediate for synthesis, in particular of peptide, by means of the present invention. When several stereogenic centres are present in the compound according to the invention, similar observations apply to the respective diastereoisomers.

Particular examples of the compounds according to the invention correspond to one of the formulae

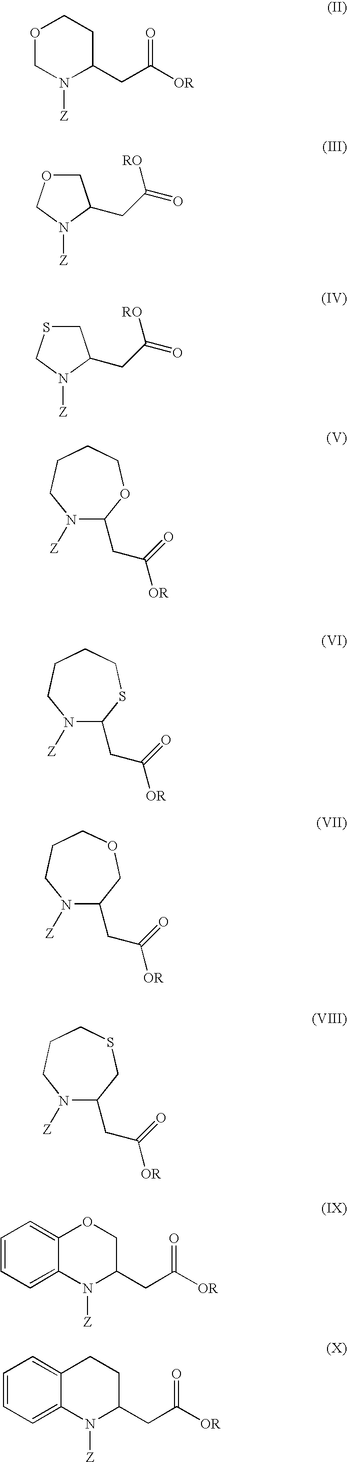

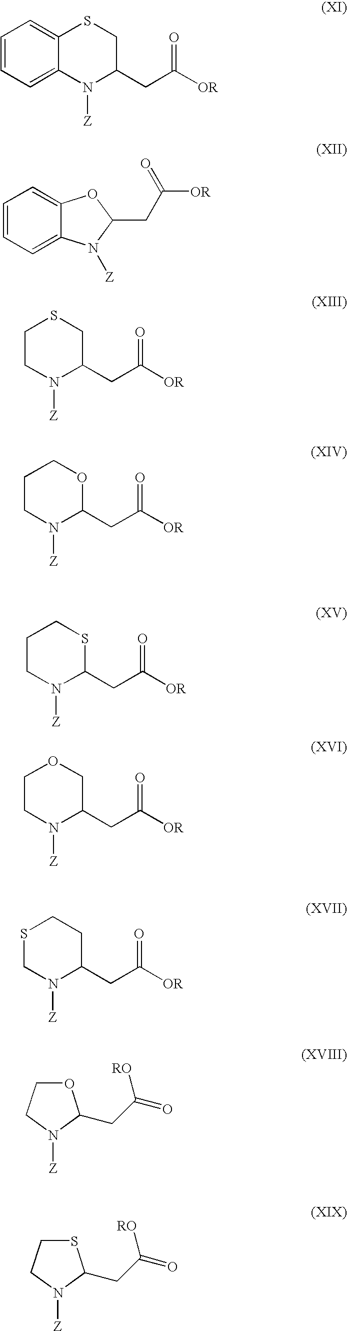

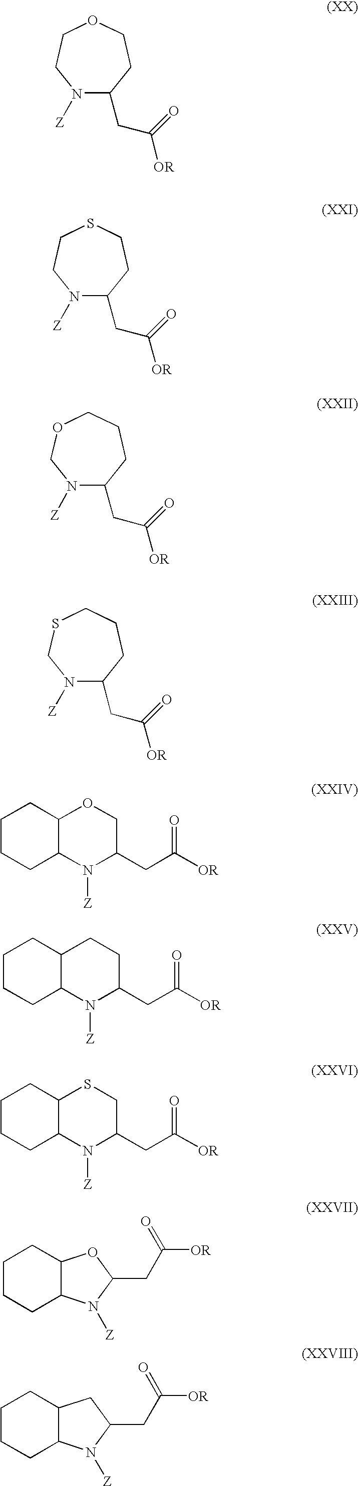

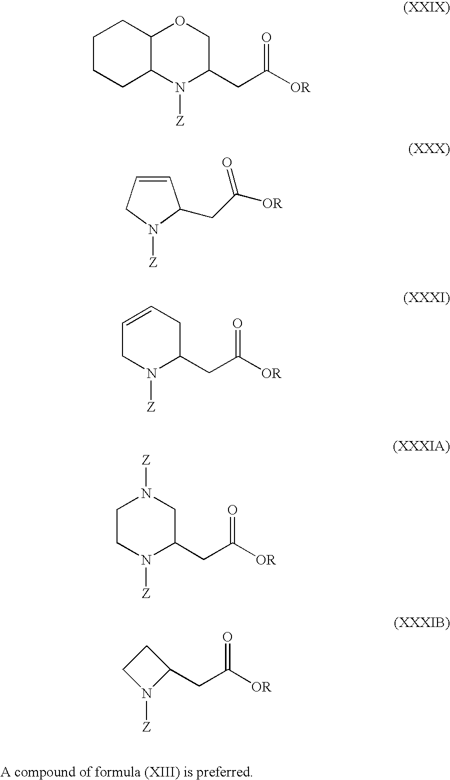

In a preferred embodiment, the compound according to the invention comprises a heterocycle corresponding to formula (XXXII) 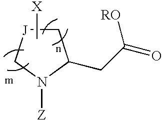
the said heterocycle being substituted with at least one substituent X. This substituent X is often chosen from a hydroxyl group, an alkoxy group, an alkyl group, an allyl group or a halogenated group.

A methoxy or ethoxy group is preferred as alkoxy group.

A methyl or ethyl group, in particular a methyl group, is preferred as alkyl group.

A fluorinated group is preferred as halogenated group. The fluorinated group is preferably chosen from —F and —CF₃.

In another preferred embodiment, X is a carbonyl group.

Particular examples of compounds according to the invention comprising a substituted heterocycle correspond to the formulae 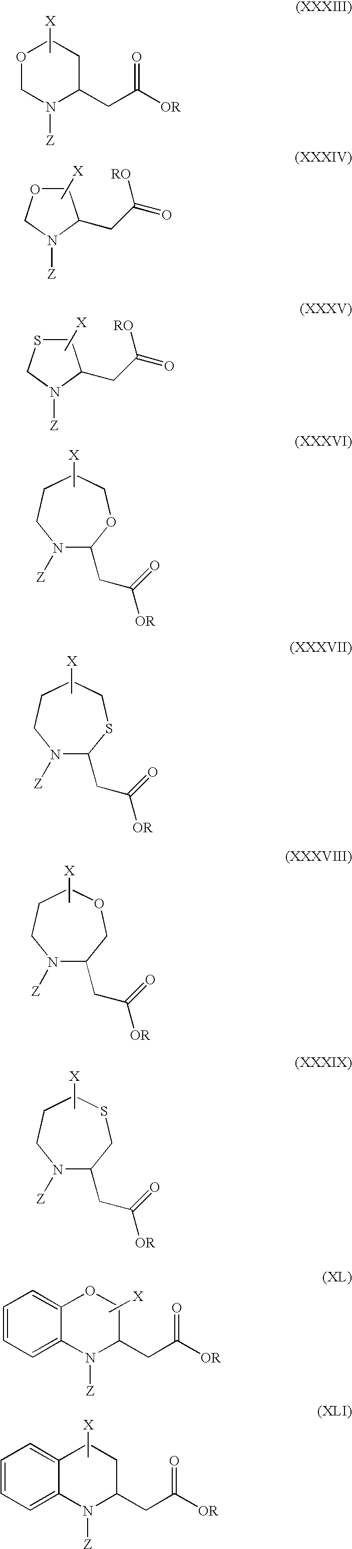 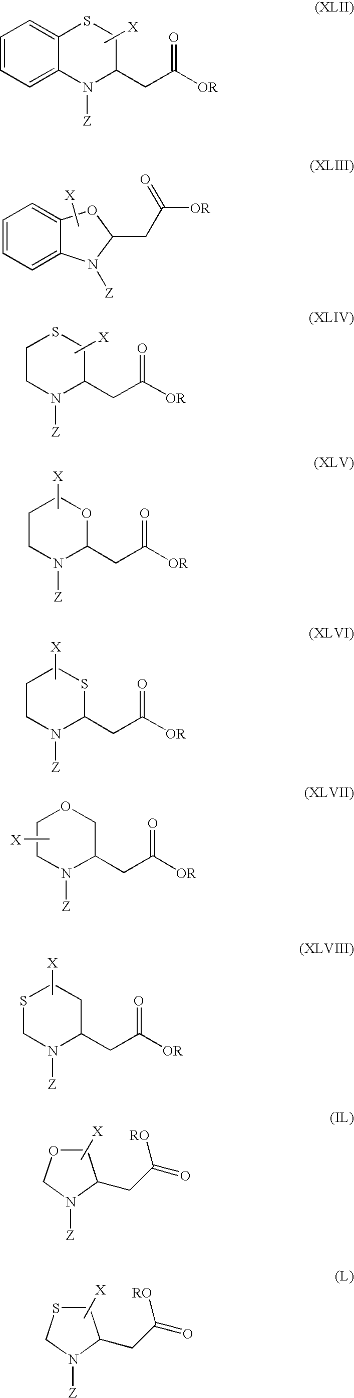 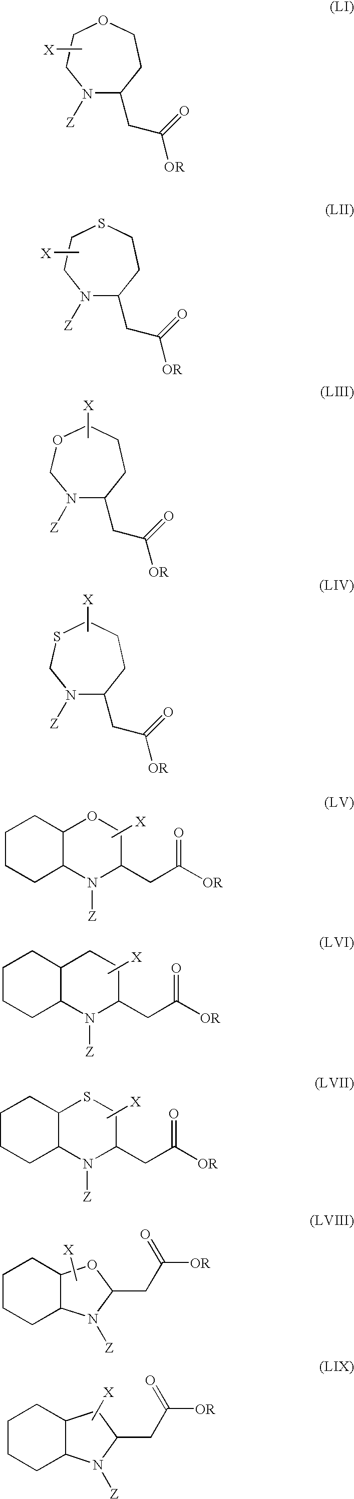 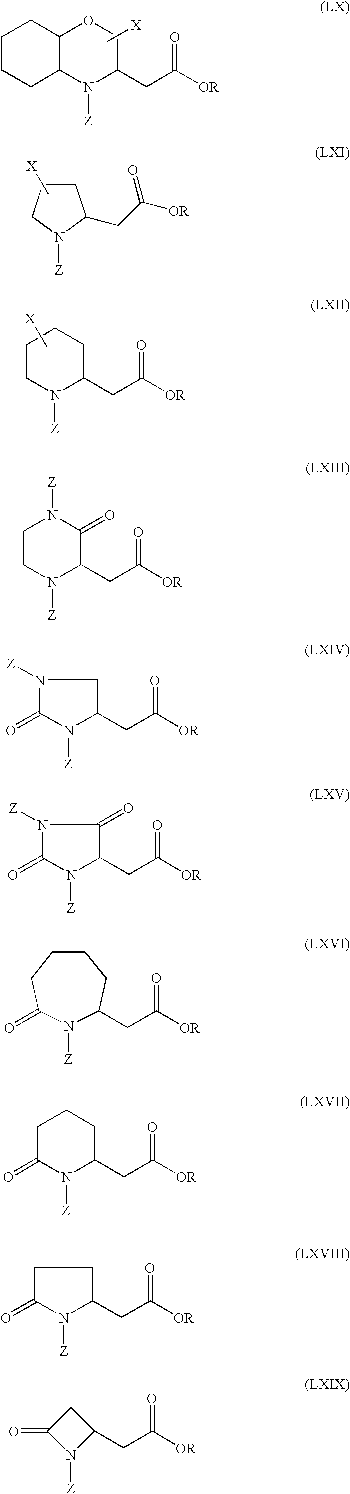
the substituents R, X and Z having the same meanings are those described above.

When the enantiopure compound corresponds to formula (XXXIII), the substituent X is often at the 2-, 5- or 6-position, preferably at the 5- or 6-position.

When the enantiopure compound corresponds to formula (XXXIV), the substituent X is often at the 2- or 5-position.

When the enantiopure compound corresponds to formula (XXXV), the substituent X is often at the 2- or 5-position.

When the enantiopure compound corresponds to formula (XXXVI), the substituent X is often at the 4-, 5-, 6- or 7-position, preferably at the 5- or 6-position.

When the enantiopure compound corresponds to formula (XXXVII), the substituent X is often at the 4-, 5-, 6- or 7-position, preferably at the 5- or 6-position.

When the enantiopure compound corresponds to formula (XXXVIII), the substituent X is often at the 2-, 5-, 6- or 7-position, preferably at the 6- or 7-position.

When the enantiopure compound corresponds to formula (XXXIX), the substituent X is often at the 2-, 5-, 6- or 7-position, preferably at the 6- or 7-position.

When the enantiopure compound corresponds to formula (XL), the substituent X is often at the 2-position.

When the enantiopure compound corresponds to formula (XLI), the substituent X often corresponds to the 3- or 4-position.

When the enantiopure compound corresponds to formula (XLII), the substituent X is often at the 2-position.

When the enantiopure compound corresponds to formula (XLIV), the substituent X is often at the 2-, 5- or 6-position, preferably at the 2- or 6-position.

When the enantiopure compound corresponds to formula (XLV), the substituent X is often at the 4-, 5- or 6-position, preferably at the 5-position.

When the enantiopure compound corresponds to formula (XLVI), the substituent X is often at the 4-, 5- or 6-position, preferably at the 5-position.

When the enantiopure compound corresponds to formula (XLVII), the substituent X is often at the 2-, 5- or 6-position, preferably at the 5-position.

When the enantiopure compound corresponds to formula (XLVIII), the substituent X is often at the 2-, 5- or 6-position, preferably at the 5-position.

When the enantiopure compound corresponds to formula (IL), the substituent X is often at the 2- or 5-position.

When the enantiopure compound corresponds to formula (L), the substituent X is often at the 2- or 5-position.

When the enantiopure compound corresponds to formula (LI), the substituent X is often at the 4-, 5-, 6- or 7-position, preferably at the 5- or 6-position.

When the enantiopure compound corresponds to formula (LII), the substituent X is often at the 4-, 5-, 6- or 7-position, preferably at the 5- or 6-position.

When the enantiopure compound corresponds to formula (LIII), the substituent X is often at the 2-, 5-, 6- or 7-position, preferably at the 5- or 6-position.

When the enantiopure compound corresponds to formula (LIV), the substituent X is often at the 2-, 5-, 6- or 7-position, preferably at the 5- or 6-position.

When the enantiopure compound corresponds to formula (LV), the substituent X is often at the 2-position.

When the enantiopure compound corresponds to formula (LVI), the substituent X is often at the 3- or 4-position.

When the enantiopure compound corresponds to formula (LVII), the substituent X is often at the 2-position.

When the enantiopure compound corresponds to formula (LVIII), the substituent X is often at the 4- or 5-position.

When the enantiopure compound corresponds to formula (LIX), the substituent X is often at the 3-, 4- or 5-position, preferably at the 3-position.

When the enantiopure compound corresponds to formula (LX), the substituent X is often at the 2-, 5- or 6-position, preferably at the 2-position.

When the enantiopure compound corresponds to formula (LXI), the substituent X is often at the 3-, 4- or 5-position, preferably at the 3- or 4-position.

When the enantiopure compound corresponds to formula (LXII), the substituent X is often at the 3-, 4-, 5- or 6-position, preferably at the 4- or 5-position.

The enantiopure compound often carries a single substituent X. It may also carry several substituents, for example in ringed compounds in which two substituents form an additional ring. Where appropriate, this additional ring may be an alicyclic, aromatic or heterocyclic ring which, may in turn be substituted with one or more substituents, in particular in accordance with the definition of substituent X.

In a first embodiment of the compounds of formula (XXXIII) to (LXII), as described above, the substituent X is a hydroxyl group which is preferably not located at the c-position with respect to the heteroatoms of the heterocycle. In this embodiment the group Z is preferably a tert-butyloxycarbonyl (BOC) group. In another preferred aspect of this embodiment, the group Z is H.

In a second embodiment of the compounds of formula (XXXIII) to (LXII), as described above, the substituent X is a fluorine (—F) group. In this embodiment, the group Z is preferably a tert-butyloxycarbonyl (BOC) group. In another preferred aspect of this embodiment, the group Z is H.

In a third embodiment of the compounds of formula (XXXIII) to (LXII), as described above, the substituent X is a methyl group. In this embodiment, the group Z is preferably a tert-butyloxycarbonyl (BOC) group. In another preferred aspect of this embodiment, the group Z is H.

In a fourth embodiment of the compounds of formula (XXXIII) to (LXII), as described above, the substituent X is a trifluoromethyl group. In this embodiment, the group Z is preferably a tert-butyloxycarbonyl (BOC) group. In another preferred aspect of this embodiment, the group Z is H.

In the compounds according to the invention and in particular in the embodiments of the compounds of formula (XXXIII) to (LXII) described above, R is preferably H.

The invention also relates to a peptide or a peptide analogue which can be obtained using a compound according to the invention in its process of manufacture. The invention also relates to a process for the manufacture of a peptide or a peptide analogue in which a compound according to the invention is used.

The peptide coupling of the compounds according to the invention may be carried out according to techniques known per se.

The invention also relates to a process for the manufacture of the enantiopure compound according to the invention, according to which
(a) a mixture of enantiomers of an ester derivative of the compound is subjected to hydrolysis in the presence of the Pseudomonas cepacia lipase; or
(b) a mixture of enantiomers of the compound, in the form of a derivative comprising at least one functional group capable of reacting with an activated carboxyl group, is subjected to a process in which
i. a reaction medium comprising the mixture of enantiomers and a reagent based on an enantiopure amino acid, in which reagent at least one amino group of the amino acid is protected with a protecting group and in which at least one carboxyl group of the amino acid is activated, is subjected to suitable conditions in order to cause the functional group capable of reacting with the activated carboxyl group to react with the activated carboxyl group so as to form a carbonyl bond;
ii. the mixture of diastereoisomers obtained is subjected to a separation operation so as to obtain at least one fraction mainly consisting of a diastereoisomer;
iii. at least part of the said fraction is subjected to a step of cleavage of the carbonyl bond under conditions in which the protecting group is essentially stable; and
iv. the enantiopure compound and optionally an enantiopure derivative of the amino acid in which at least one amino group is protected with the protecting group are recovered.

The feature (a) of the process of manufacture of the compound according to the invention may be preferably carried out according to the method and in particular under the conditions described in patent applications FR 03.04219 and PCT/EP2004/003688 in the name of the applicant, the content of which is incorporated by reference into the present application.

The feature (b) of the process of manufacture of the compound according to the invention may be preferably carried out according to the method and in particular under the conditions described in patent applications FR 03.10582 and PCT/EP2004/052094 in the name of the applicant, the content of which is incorporated by reference into the present application.

The racemic derivatives of the compound according to the invention may be obtained starting with the corresponding heterocycles not substituted at the α-position in relation to the nitrogen, for example by a reaction sequence comprising
(a) electrochemical methoxylation of an n-acylated derivative of the said heterocycle;
(b) allylation of the N^{α}-methoxylated derivative, for example of allyltrimethylsilane in the presence of a catalyst such as TiCl₄;
(c) oxidative cleavage, for example, by ozonolysis of the allyl bond.

The examples below are intended to illustrate the invention without however limiting it.

### EXAMPLE 1

### Resolution of 4-tert-butoxycarbonyl-3-carbomethoxymethyl-thiomorpholine

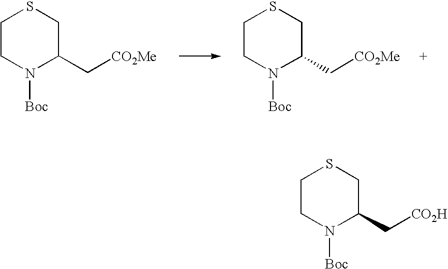

Racemic 4-tert-butoxycarbonyl-3-carbomethoxymethyl-thiomorpholine was obtained by reacting N-Boc-2-aminoethanethiol with methyl-4-bromocrotonate and diisopropylethylamine in THF at 0° C. The solution was agitated for 24 H at room temperature. THF was evaporated and the residue was taken up in dichloromethane. This organic phase was washed with 5% NaCl-solution and trifluoroacetic acid was added until the N-Boc function had been deprotected. Dichloromethane was evaporated and the residual material was diluted in toluene without prior purification. Triethylamine was added and the solution was heated. After the reaction, toluene was evaporated and the residue was dissolved in dioxane/water mixture and protected with Boc₂O in presence of LiOH.

300 mg of Pseudomonas cepacia PS Amano lipase were added to a solution of 551 mg of β-amino ester (2.0 mmol) in 10 ml of water, 2 ml of buffer pH 7 (10⁻¹M) and 2 ml of THF. The temperature was maintained at 20° C. and the pH was maintained at 7 by addition of a 0.1 N sodium hydroxide solution. After adding 8.5 ml of 0.1 N sodium hydroxide and stirring for 3 days, the solution was filtered. It was then concentrated and the aqueous phase was then extracted with 3 times 10 ml of ether. The organic phases were combined and dried on magnesium sulphate. After evaporation, 220 mg of ester were obtained (yield: 40%; ee>99%). The aqueous phase was acidified to pH 3 and then extracted with 3 times 10 ml of ether. The organic phases were combined and dried on magnesium sulphate. After evaporation, 220 mg of 4-tert-butoxycarbonyl-3-carboxymethylthiomorpholine were obtained (yield=42%; ee>98%).

### (3R)-4-tert-Butoxycarbonyl-3 -carboxymethylthiomorpholine

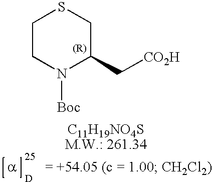

¹H NMR (500 MHz):

δ ppm (CD₃OD): 1.48 (s, 9H); 2.44 (d, J=13 Hz, 1H); 2.55 (d, J=13.7 Hz, 1H); 2.67 (td, J=3.3 and 13.3 and 12.6 Hz, 1H); 2.86 (m, 2H); 2.99 (dd, J=3.8 and 13.9 Hz, 1H); 3.15 (m, 1H); 4.23 (d, J=13.1 Hz, 1H); 4.88 (m, 1H).

### (3S)-4-tert-Butoxycarbonyl-3-carbomethoxymethylthiomorpholine

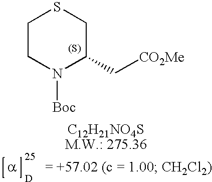

¹H NMR (500 MHz):

δ ppm (CD₃OD): 1.47 (s, 9H); 2.44 (d, J=13.2 Hz, 1H); 2.53 (d, J=13.8 Hz, 1H); 2.67 (td, J=3.3 and 13.3 and 12.5 Hz, 1H); 2.92 (m, 2H); 2.98 (dd, J=3.8 and 13.9 Hz, 1H); 3.15 (m, 1H); 3.68 (s, 3H); 4.22 (d, J=12.8 Hz, 1H); 4.88 (m, 1H)

### EXAMPLE 2

### Synthesis of L-N-methyl-Phe-(3S)-3-carbomethoxymethylthiomorpholine

L-N-methyl-Phe-(3S)-3-carbomethoxymethylthiomorpholine is obtained by coupling L-N-methyl-Phe-OH with (3S)-3-carbomethoxymethylthiomorpholine which can be obtained by deprotection of the N-Boc derivative with trifluoroacetic acid in presence of isobutylchloroformiate as activator for the carboxylic function.

### EXAMPLE 3

### Synthesis of N-BOC-β-homopyroglutamic acid

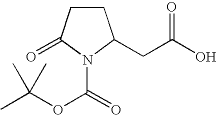

Racemic N-boc-β-homopyroglutamic acid was obtained starting from pyroglutamic acid by electrochemical reaction in methanol, followed by allylation with allyltrimethylsilane catalyzed by titanium tetrachloride, protection with tert.butoxycarbonic acid anhydride and oxidation with RuCl₃/NaIO₄ in overall yield of 50% based on pyroglutamic acid.

The racemic acid was esterified with dicyclohexylcarbodiimide in a mixture of methanol and methylene chloride to obtain corresponding racemic methyl ester.

Following an analogous procedure to example 1, ester having an 95% e.e determined by GC and acid (LXXa) having an 96% e.e determined by GC were obtained

The ester can be saponified to give acid (LXXb).

### EXAMPLE 4

### Synthesis of Piperazine Derivative

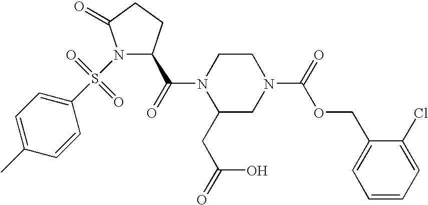

Racemic piperazine derivative was obtained starting from dibenzylethylenediamine by addition of methyl-4-bromocrotonate and triethylamine in toluene at 0° C. The solution was agitated for 48 H at room temperature. After evaporation, the medium was hydrolyzed with 10% HCl and extracted with ethyl acetate. The aqueous phase was basified with K₂CO₃ until pH 7, and extracted with ethyl acetate. The organic phase was evaporated to give oil, followed by hydrogenation catalyzed by palladium on carbon in a mixture methanol-HCl 1N, the corresponding product was obtained after filtration on celite and evaporation. A selective protection with 2-chlorobenzyloxysuccinimide was achieved at pH 8.5 regulating pH with K₂CO₃ (2M) at 0° C. Then the aqueous phase was acidified with KHSO₄ 5% until pH 2.5 and extracted with ethyl acetate. The organic phase was evaporated to give yellow oil.

The free amine functionality was coupled with (2S)-1-tosylpyroglutamyl chloride in water/dioxane in presence of Na₂CO₃. After approximately 8 h, the reaction was stopped. The medium was extracted with ethyl acetate, and the organic phase was evaporated to give a solid. After saponification, the mixture of diastereomers was separated by HPLC to give tosylated derivatives (R) (LXXIa)- and (S) (LXXIb) respectively.

### EXAMPLE 5

### Synthesis of Oxygenated Piperazine Derivative

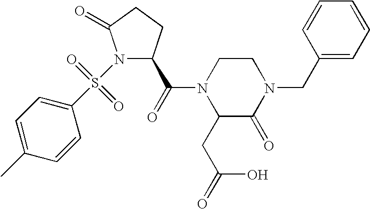

Racemic oxygenated piperazine derivative was obtained starting from monobenzylethylenediamine by addition of dimethylmaleate in methanol, the mixture was agitated for 24 h, and the medium was evaporated to give yellow oil. The free amine functionality was coupled with (2S)-1-tosylpyroglutamyl chloride in water/dioxane in presence of Na₂CO₃. After approximately 8 h, the reaction was halted. The medium was extracted with ethyl acetate, and the organic phase was evaporated to give a solid. After saponification, the mixture of diastereomers is separated by HPLC.

The free amine functionality can be coupled with pTos-Glp-Cl according to the same procedure as described in example 4 and similar enantiomeric separation and yield are obtained to give the tosylated derivatives (R) (LXXIIa)- and (S) (LXXIlb).

### EXAMPLE 6

### Synthesis of Peptide Based on Compounds of Examples 3-5

Persilylated Phe-OH which can be obtained by reacting phenylalanine with trimethylsilylcyanide in the presence of triethylamine is reacted with compounds of examples 3-5 in the presence of isobutylchloroformiate for activation of the carboxylic function.

The dipeptides
(LXXa)-Phe
(LXXb)-Phe
(LXXIa)-Phe
(LXXIb)-Phe
(LXXIIa)-Phe
(LXXIIb)-Phe
   are obtained in good yields

In a similar manner, the compounds of examples 3 to 5 can be reacted with persilylated glycine. The corresponding dipeptides
(LXXa)-Gly
(LXXb)-Gly
(LXXIa)-Gly
(LXXIb)-Gly
(LXXIIa)-Gly
(LXXIIb)-Gly
   are obtained in good yields

### EXAMPLE 7

### Synthesis of Peptidomimetic

The N-benzyl-group of LXXIIa-Phe which can be obtained according to the procedure described in example 6 is deprotected by hydrogenolysis. The deprotected peptide is persilylated and coupled with N-acetyl-Ala to give the corresponding peptidomimetic.

### EXAMPLE 8

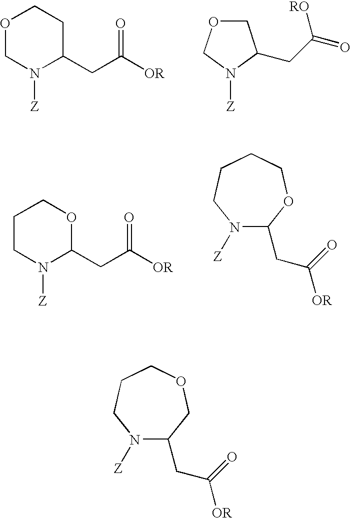

The racemic esters of compounds indicated in example 8, wherein Z=BOC and R=methyl can be obtained starting from the corresponding N-protected unsubstituted heterocycle according to the procedure described in the example 3, with the difference that oxidative ozonolysis is used instead of RuCl₃/NaIO₄ oxidation. The racemic esters can be separated with lipase as described in example 1.

### EXAMPLE 9

### Synthesis of Peptide Based on Compounds of Example 8

The enantiopure esters obtained in example 8 can be coupled with N-Boc-Phe according to the technique of example 2 to give corresponding dipeptides in good yield.

1. An enantiopure heterocyclic compound of formula (I) 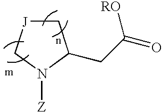
in which J is C, N, O or S; Z is H or a group for protecting the amino functional group, R is H or an organic residue, m is 0, 1 or 2 and n is 0, 1 or 2, and in which the heterocycle is optionally substituted with at least one substituent other than CH₂—COOR

2. The compound according to claim 1, in which J is O or S.

3. The compound according to claim 1, in which m is 1 or 2 and n is 0 or 1.

4. The compound according to claim 1, corresponding to one of the formulae

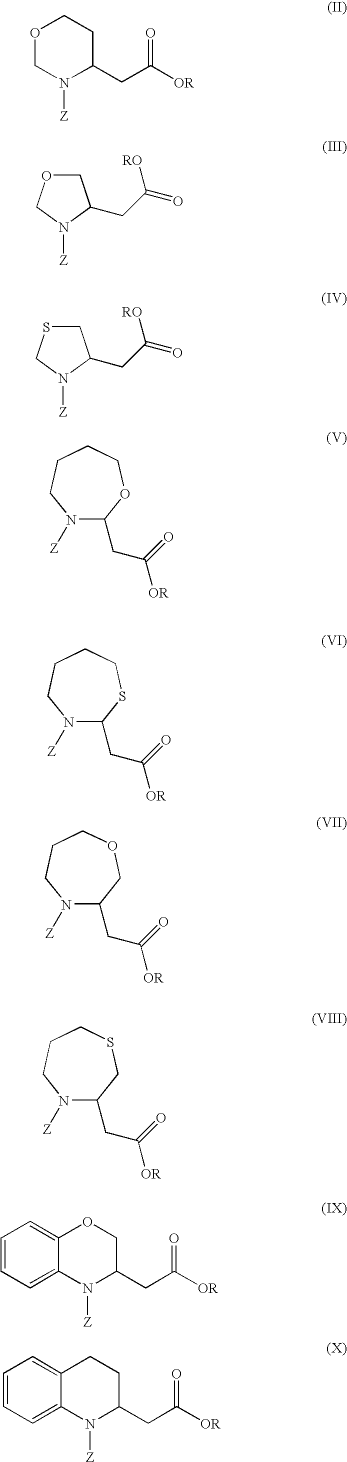

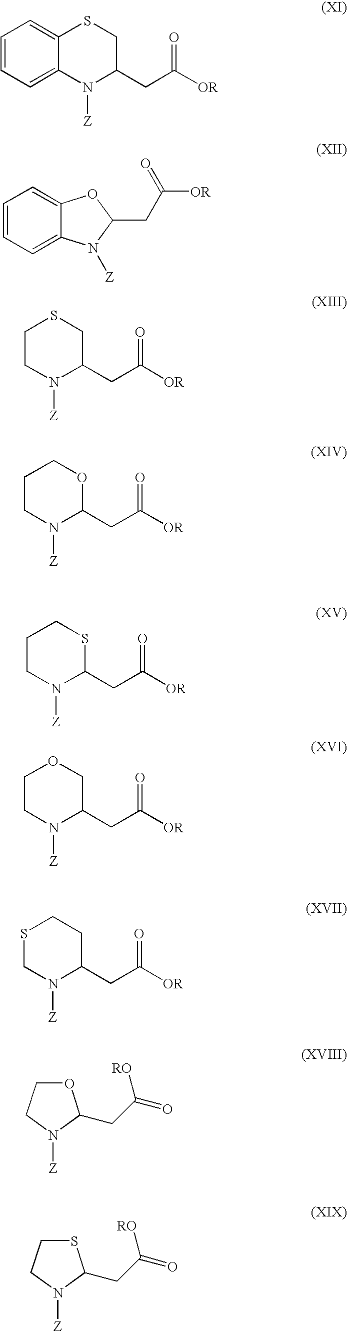

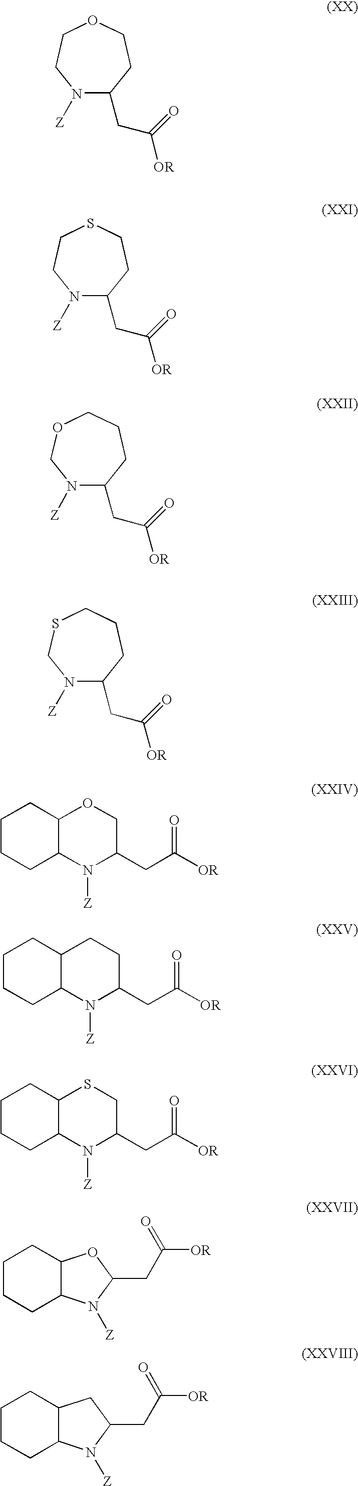

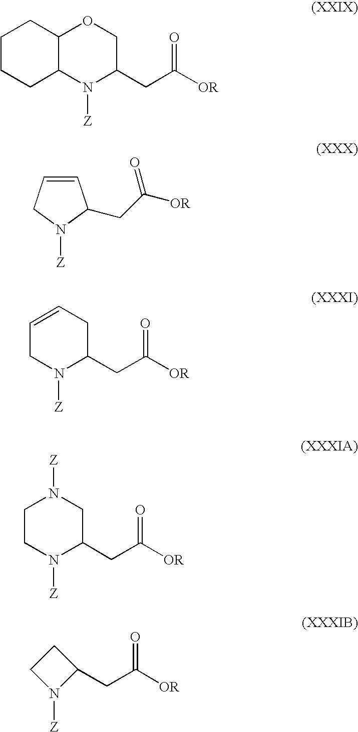

5. The compound according to claim 4, corresponding to formula

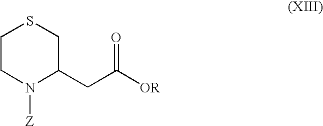

6. The compound according to claim 1, corresponding to one of the formulae 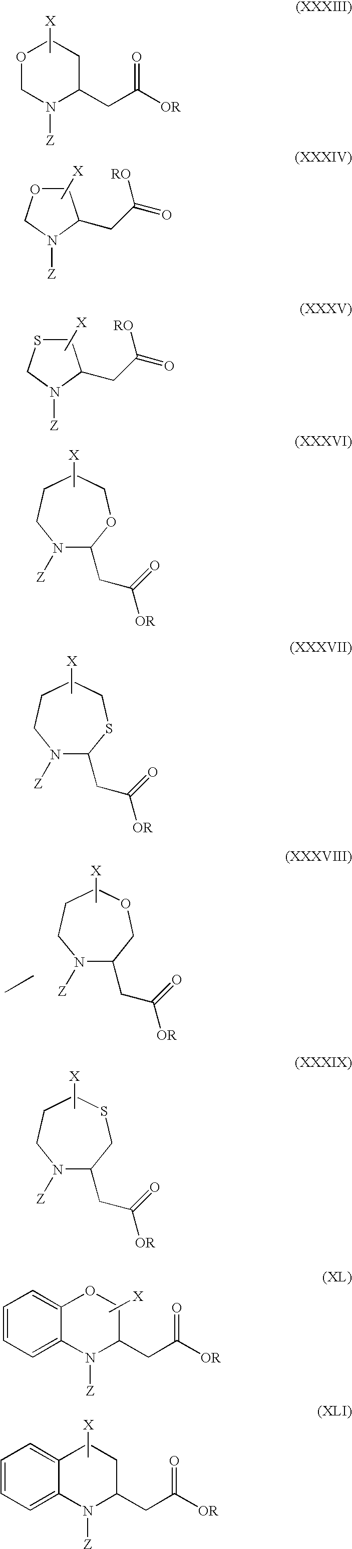 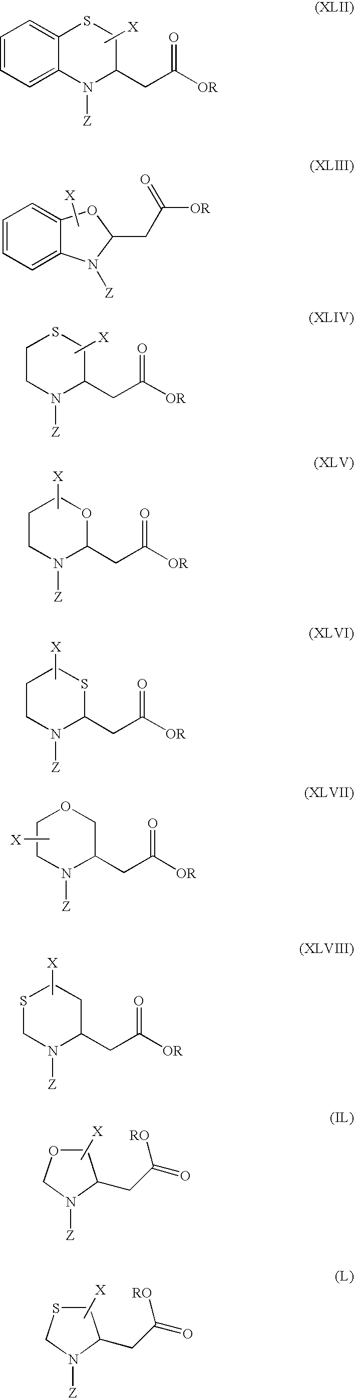 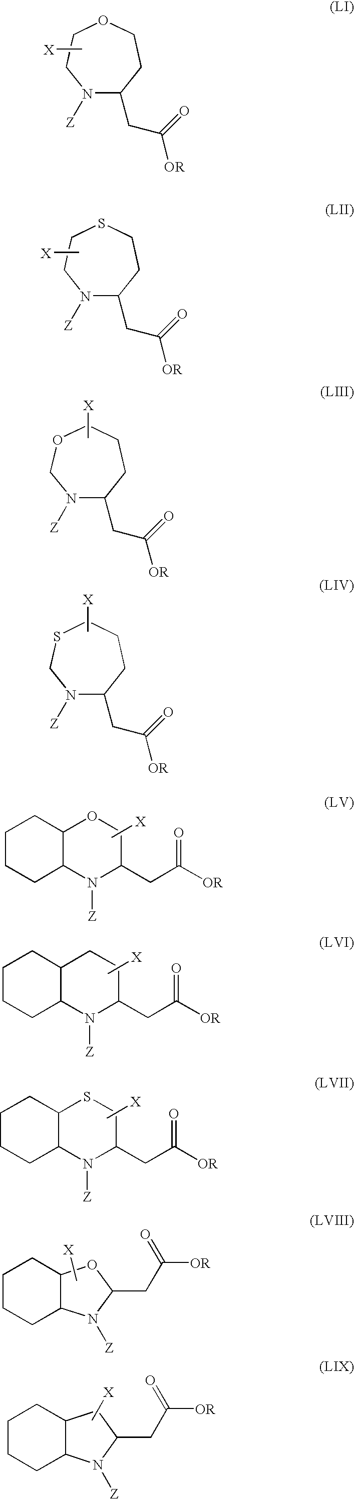 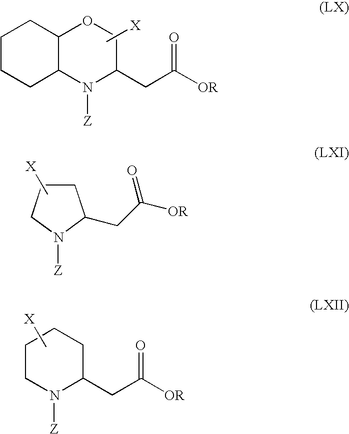
and in which X denotes a substituent.

7. The compound according to claim 1, in which the heterocycle is substituted with at least one substituent selected from the group consisting of a hydroxyl group, an alkyl group, an allyl group and a halogenated group.

8. The compound according to claim 7, in which the halogenated group is a fluorinated group.

9. The compound according to claim 1, corresponding to one of the formulae

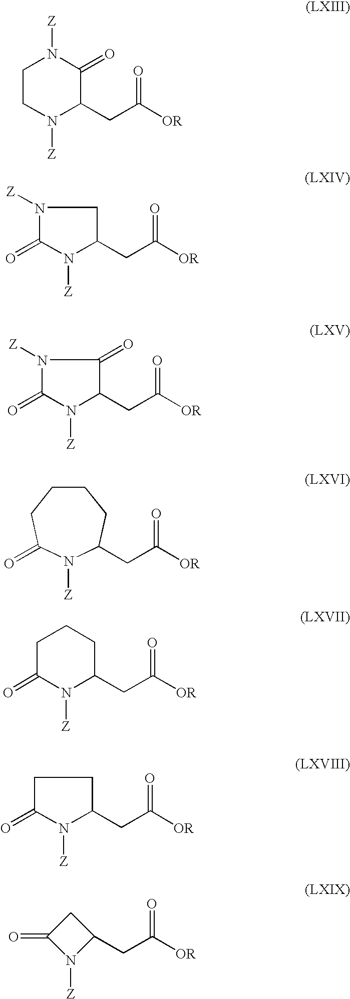

10. The compound according to claim 1, in which the substituent Z is an alkoxycarbonyl group, an aryloxycarbonyl group or an aralkoxycarbonyl group.

11. The compound according to claim 10, in which the substituent Z is a tert-butyloxycarbonyl (BOC) group.

12. A process for the manufacture of the enantiopure compound according to claim 1 comprising the steps of
(a) hydrolyzing a mixture of enantiomers of an ester derivative of said compound in the presence of Pseudomonas cepacia lipase; or
(b) a mixture of enantiomers of said compound, in the form of a derivative comprising at least one functional group capable of reacting with an activated carboxyl group, is subjected to the process comprising the steps of
i. reacting said mixture of enantiomers and a reagent based on an enantiopure amino acid, in which reagent at least one amino group of the amino acid is protected with a protecting group and in which at least one carboxyl group of the amino acid is activated, in a reaction medium under suitable conditions in order to cause the functional group capable of reacting with the activated carboxyl group to react with the activated carboxyl group so as to form a carbonyl bond;
ii. separating the mixture of diastereoisomers obtained so as to obtain at least one fraction mainly consisting of a diastereoisomer;
iii. cleaving the carbonyl bond of the diastereoisomer in at least part of said fraction under conditions in which the protecting group is essentially stable; and
iv. recovering said enantiopure compound and optionally an enantiopure derivative of the amino acid in which at least one amino group is protected with the protecting group.

13. A peptide or peptide analogue which can be obtained using a compound according to claim 1 in its process of manufacture.

14. The compound according to claim 8, in which the halogenated group is —F or —CF₃.
